(19) United States
(12) Patent Application Publication
(10) Pub. No.: US 2021/0371803 A1
(43) Pub. Date: Dec. 2, 2021

(54) MULTI-PARAMETER AUTOMATIC BLOOD CELL COUNTING DEVICE

(71) Applicant: Shimbu So, Tokyo (JP)

(72) Inventor: Shimbu So, Tokyo (JP)

(21) Appl. No.: 16/943,483

(22) Filed: Jul. 30, 2020

(30) Foreign Application Priority Data
Jun. 2, 2020 (JP) 2020-095973

(51) Int. Cl. C12M 1/34; G01N 33/569; G01N 33/50; G01N 15/12

(52) U.S. Cl. CPC C12M 41/36; G01N 33/56966; G01N 2015/1062; G01N 15/12; G01N 2800/24; G01N 33/5091

(57) ABSTRACT

A multi-parameter automatic blood cell counting device includes a basic information generating part, an immune cell subgroup detection part, an immunodynamic change analyzing part, a storage part, and display part. The basic information generating part acquires examination information about a blood count of blood components and a white blood cell image and CD classification analysis information about monoclonal antibodies that bind to lymphocyte surface antigens. The immune cell subgroup detection part detects information about immune cell subgroups required for analyzing an immune status of a subject based on the examination information and the CD classification analysis information. The immunodynamic change analyzing part identifies information about the change in immunodynamics of the subject based on the information detected by the immune cell subgroup detection part and the examination information and CD classification analysis information stored in the storage part, and displays an image of the identified information on the display part.

### CROSS-REFERENCE TO RELATED APPLICATION

The present application is based on and claims a priority benefit of Japanese patent application No. 2020-095973, filed on Jun. 2, 2020, the disclosure of which is hereby incorporated herein by reference in its entirety.

### BACKGROUND

This disclosure relates to a multi-parameter automatic blood cell counting device that can perform follow-up examination on a change in immunodynamics.

Currently, in the clinical field, there are many cases in which immunodynamics are examined by examining the interrelationship between lymphocytes. When the dynamics of immune subgroups are examined, it is possible to predict autoimmune diseases, allergic diseases, the presence of cancer, transition to cancer status and detailed cancer information. In addition, the examination of immunodynamics is not only beneficial for early detection of diseases but can also be used for evaluation indexes for therapeutic effects and side effects in prevention and treatments of diseases, and has become an essential examination item for early detection and early treatment of diseases in the modern medical field.

It is needless to say that helper T cells which are called a control tower of immparty are basic indicators in examination of immunodynamics, but additionally indicator information about killer T cells, B cells, NK cells, NKT cells, and the like is also important. For example, based on the above basic indicators, it is possible to derive detailed information having strong relations with immune symptoms with Th1 cells, Th2 cells, Th17 cells, regulatory T cells, and the like, and this is beneficial for early detection and early treatment of diseases.

On the other hand, there are two blood sampling tests (for example, in Japan): an in-hospital test (general examination item) and an out-of-hospital test performed in an examination facility and the like (an examination including items in JP2008-89382A). Four types of fraction of granulocytes (neutrophils, eosinophils, basophils, and mononuclear cells) and an overall examination report of lymphocytes have been mainstream for a white blood cell clinical examination (in-hospital test) since 40 years ago. In these clinical examinations, fractions of the dynamic state of granulocytes are shown, but fractions of the states of lymphocyte subgroups are not shown (for example, refer to the table on the left in FIG. 2). Currently, showing the fractions of the states of lymphocyte subgroups (for example, refer to the table on the right in FIG. 2), as disclosed in, for example, JP2008-89382A, is very complicated and troublesome, and examination of individual blood collection tubes needs to be performed.

In the related art, in order for many medical institutions to obtain information about a more detailed immune status (information about the fractions of the dynamic state of granulocytes and the fractions of the dynamic state of lymphocyte subgroups), it is necessary to request an examination at a specialized institution such as Tokyo Koshueisei Laboratories, Inc., and it is necessary to perform an individual blood collection tube examination (for example, a complicated method such as a PCR method) in a custom-made manner, which is very expensive. In particular, in order to investigate the dynamic state of lymphocyte subgroups, since it is necessary to examine a large number of items simultaneously, it is very unreasonable to perform an examination by a conventional technique in the clinical stage in which individual items are required. In addition, according to complicated and expensive examination methods up to now, information about many types of items is indeed obtained, but all of them are merely static examinations for specific parts of immparty. That is, in the clinical field, it is not possible to obtain indicator information about helper T cells, killer T cells, B cells, NK cells, NKT cells, and the like immediately, and it is difficult to obtain information about the dynamics of the entire lymphocyte subgroups, which cause major problems for healthcare staff and patients.

As shown in FIG. 13, indicator information (some of immune cell subgroups) about helper T cells, killer T cells, B cells, NK cells, NKT cells, and the like is directly related to many pathological conditions and constitutional changes (changes in cells). That is, when indicator information about helper T cells, killer T cells, B cells, NK cells, NKT cells, and the like is immediately acquired, it is possible to determine and cope with many pathological conditions and constitutional changes early on.

In recent years, a blood cell count examination device that counts blood cells in a blood sample and measures a hemoglobin concentration has become known in which, particularly in this type of device, in order to realize a small examination device for point of care testing (POCT: simple quick examination), a blood cell count sensor, a hemoglobin concentration measurement sensor, a blood sample volume weighing sensor and the like, which are main parts thereof, are reduced in size and integrated to obtain a blood cell count examination chip having a disposable structure for the device main body, and the blood cell count examination chip is used in the blood cell count examination device (refer to JP2008-89382A). In addition, an immparty evaluation method, an immparty evaluation device, and an immparty evaluation program through which an immparty is evaluated using immune cell markers corresponding to a plurality of immune cells contained in collected blood, and an information recording medium in which the immparty evaluation program is recorded are known (refer to WO2007/145333A1). However, a device that can immediately acquire indicator information about helper T cells, killer T cells, B cells, NK cells, NKT cells, and the like is not yet known.

In modern society, in order to prevent the spread of infectious diseases due to unknown viruses such as the rapid spread of infectious diseases due to unknown viruses resulting from the increasing movement of people and globalization, it is important to immediately determine the immune status of people and find actual cases. In the modern clinical field, device machines such as an automatic blood cell measuring device and a flow cytometer are used, but it is necessary to perform a complicated method such as a PCR method in order to ascertain information about Th1 cells, Th2 cells, Th17 cells, regulatory T cells and the like which are strongly related to immune symptoms. In addition, information about related cells obtained by a complicated method such as a PCR method is information indicating a static state and it is difficult to obtain information about immunodynamics. Therefore, for clinical staff, the development of a device in which a complicated method such as a PCR method is not performed regarding information about Th1 cells, Th2 cells, Th17 cells, regulatory T cells and the like which are strongly related to immune symptoms, and information about immunodynamics is displayed at an earlier stage is required.

### SUMMARY

In view of the above circumstances, an object of the present disclosure is to allow medical staff to immediately display immparty information about examinees and specifically information about immunodynamics.

### BRIEF DESCRIPTION OF DRAWINGS

FIG. 1 is a schematic view of a multi-parameter automatic blood cell counting device according to a first embodiment.

FIG. 2 is a diagram showing fractions of statuses of lymphocyte subgroups.

FIG. 3A is a diagram showing an immunodynamic pattern formed by an immunodynamic change analyzing part according to the first embodiment.

FIG. 3B is a diagram showing an immunodynamic model formed by the immunodynamic change analyzing part according to the first embodiment.

FIG. 4 is a schematic view of the immunodynamic change analyzing part according to the first embodiment.

FIG. 5 shows diagrams of proportions of the number of CD4 antigens, the number of CD8 antigens, the number of NK cells, the number of NKT cells and the number of B cells of infected persons displayed on a display part.

FIG. 6A is a diagram showing comparison influenza infection pattern standard data that is displayed on the display part by a pattern formation module.

FIG. 6B is a diagram showing immune cell basic data of a subject that is displayed on the display part by the pattern formation module.

FIG. 7 shows a table in which components of patients with gastric cancer and healthy persons are compared, (A) is a pie chart showing proportions of components of the white blood cell subgroups of patients with gastric cancer, (B) is a pie chart showing proportions of only the lymphocyte subgroups of patients with gastric cancer, (C) is a pie chart showing proportions of components of the white blood cell subgroups of healthy persons, and (D) is a pie chart showing the proportion of only the lymphocyte subgroups of healthy persons.

FIG. 8 shows a table in which components of patients with rheumatoid arthritis and patients with atopic dermatitis are compared, (A) is a pie chart showing proportions of components of the white blood cell subgroups of patients with rheumatoid arthritis, (B) is a pie chart showing proportions of the lymphocyte subgroups and single cells of patients with rheumatoid arthritis, (C) is a pie chart showing the proportion of only the lymphocyte subgroups of patients with rheumatoid arthritis, (D) is a pie chart showing proportions of components of the white blood cell subgroups of patients with atopic dermatitis, (E) is a pie chart showing proportions of the lymphocyte subgroups and single cells of patients with atopic dermatitis, and (F) is a pie chart showing the proportion of only the lymphocyte subgroups of patients with atopic dermatitis.

FIG. 9 shows a table in which components of patients with esophageal candida and patients with psoriasis are compared, (A) is a pie chart showing proportions of components of the white blood cell subgroups of patients with esophageal candida, (B) is a pie chart showing the proportion of only the lymphocyte subgroups of patients with esophageal candida, (C) is a pie chart showing proportions of components of the white blood cell subgroups of patients with psoriasis, and (D) is a pie chart showing the proportion of only the lymphocyte subgroups of patients with psoriasis.

FIG. 10 is a diagram showing an immunodynamic model formed by an immunodynamic correlation part of the first embodiment.

FIG. 11 shows a table in which components in a subject before NK cells are administered and in the subject after NK cells are administered are compared, (A) is a pie chart showing proportions of components of the white blood cell subgroups of the subject before NK cells are administered, (B) is a pie chart showing proportions of monocytes and lymphocyte subgroups of the subject before NK cells are administered, (C) is a pie chart showing the proportion of only the lymphocyte subgroups of the subject before NK cells are administered, (D) is a pie chart showing proportions of components of the white blood cell subgroups of the subject after NK cells are administered, (E) is a pie chart showing proportions of monocytes and lymphocyte subgroups of the subject after NK cells are administered, and (F) is a pie chart showing the proportion of only the lymphocyte subgroups of the subject after NK cells are administered.

FIG. 12 is a diagram showing an immunodynamic model showing an immparty correlation from an influenza infection pattern 1 (during influenza infection) to an influenza recovery pattern 1 (during influenza recovery).

FIG. 13 is a schematic view showing pathological conditions and changes in cells which are related with immune cell subgroups.

### DETAILED DESCRIPTION

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

Hereinafter, embodiments of a multi-parameter automatic blood cell counting device 1 according to the present disclosure will be described with reference to the drawings.

### First Embodiment

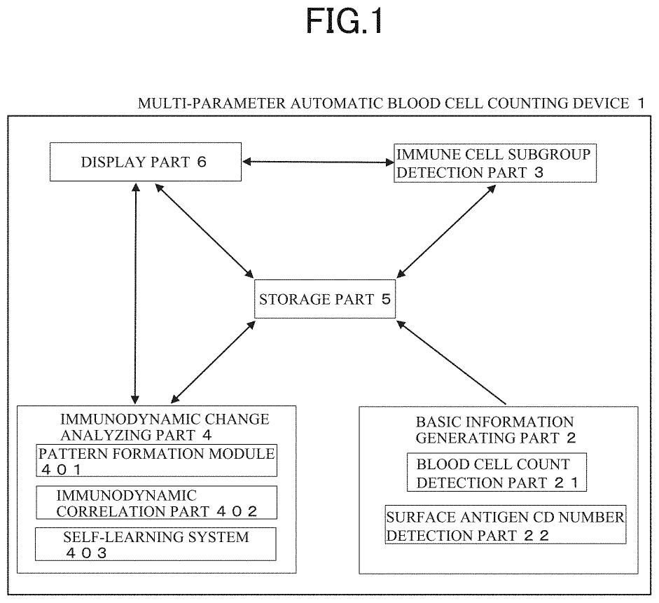

As shown in FIG. 1, the multi-parameter automatic blood cell counting device 1 of a first embodiment includes a basic information generating part 2, an immune cell subgroup detection part 3, an immunodynamic change analyzing part 4, a storage part 5 and a display part 6.

The basic information generating part 2 generates the number of white blood cells, a white blood cell image (white blood cell fraction information), an analysis result of a surface antigen CD number of white blood cells (immune cells), and the like.

In the present embodiment, the basic information generating unit 2 acquires the number of white blood cells, a white blood cell image (white blood cell fraction information), and an analysis result of a surface antigen CD number of white blood cells (immune cells). As shown in FIG. 1, the basic information generating unit 2 is composed of a blood cell count detecting unit 21 and a surface antigen CD number detecting unit 22.

In the present embodiment, the blood cell count detecting unit 21 is composed of a blood cell counting device that performs treatments such as dilution, hemolysis, and staining of blood and optically or electrically counts blood cells or a blood cell count receiver that receives blood cell counts from other blood cell counting devices. That is, the blood cell count detecting unit 21 may acquire the number of white blood cells and a white blood cell image (white blood cell fraction information).

When the blood cell count detecting unit 21 is a blood cell counting device, the blood cell count detecting unit 21 analyzes blood components, and detects the number of white blood cells, and a proportion (%) of basophils, a proportion (%) of eosinophils, a proportion (%) of neutrophils, a proportion (%) of lymphocytes, and a proportion (%) of monocytes in white blood cells.

When the blood cell count detection part 21 is a blood cell count receiver, the blood cell count detection part 21 receives (acquires) only information about the number of white blood cells, a proportion (%) of basophils, a proportion (%) of eosinophils, a proportion (%) of neutrophils, a proportion (%) of lymphocytes, and a proportion (%) of monocytes in white blood cells from an existing blood cell count detection part, a server, and the like.

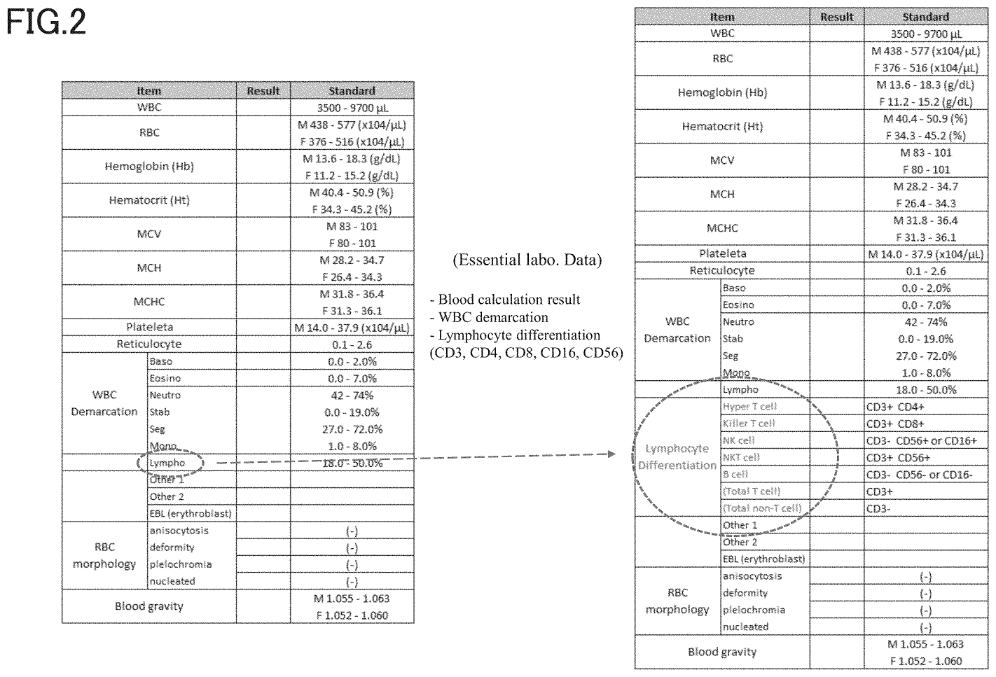

An example of information that is generated or received by the blood cell count detection part 21 will be described with reference to a list of examination results of a blood count of blood components and a white blood cell image of a general subject shown in FIG. 2. In FIG. 2, “WBC” indicates the number of white blood cells in 1 μl, and in items of a white blood cell image (white blood cell fraction information), “Baso” indicates a proportion (%) of basophils in white blood cells, “Eosino” indicates a proportion (%) of eosinophils, “Neutro” indicates a proportion (%) of neutrophils, “Lympho” indicates a proportion (%) of lymphocytes, and “Mono” indicates a proportion (%) of monocytes. That is, a blood cell count receiver 212 is required to generate or receive only the number of white blood cells, a proportion (%) of eosinophils, a proportion (%) of neutrophils, a proportion (%) of lymphocytes, and a proportion (%) of monocytes.

The surface antigen CD number detection part 22 is composed of, for example, a flow cytometer that detects CD3 antigens, CD56 antigens (and/or CD16 antigens), CD4 antigens and CD8 antigens, or a receiver for an analysis result of a surface antigen CD number of white blood cells, which receives these pieces of information.

When the surface antigen CD number detection part 22 is a flow cytometer, the surface antigen CD number detection part 22 may be any part including a flow cell, a light source, a detector, a signal amplification system part, a conversion system part, and the like, and is preferably a part that does not include a plurality of photodetectors corresponding to a plurality of types of fluorescence detection.

However, the flow cytometer may detect only CD3 antigens, CD56 antigens (and/or CD16 antigens), CD4 antigens and CD8 antigens.

When the surface antigen CD number detection part 22 is a CD number analysis result receiver, the surface antigen CD number detection part 22 may receive (acquire) only information about CD3 antigens, CD56 antigens (and/or CD16 antigens), CD4 antigens and CD8 antigens from an existing analyzing device, a server, and the like.

The storage part 5 is composed of hardware (circuits, dedicated logics, and the like), software (such as that operating in a general-purpose computer system or a dedicated machine), and like and has a function of storing various types of information. In the storage part 5, analysis information obtained by detecting or analyzing in the basic information generating part 2, the immune cell subgroup detection part 3 and the immunodynamic change analyzing part 4 and the like are stored.

The display part 6 has a function of displaying analysis information obtained by detecting or analyzing in the basic information generating part 2, the immune cell subgroup detection part 3 and the immunodynamic change analyzing part 4. Regarding the display part 6, a general liquid crystal display such as a touch panel or a display panel can be used.

The immune cell subgroup detection part 3 detects a total number of helper T cells and a total number of killer T cells in the lymphocyte subgroups (a total number of T cells in the lymphocyte subgroups), a total number of B cells, a total number of NK cells and a total number of NKT cells based on the number of white blood cells and an analysis result (analysis information) of a surface antigen CD number of white blood cells (immune cells) acquired in the basic information generating part 2. Regarding T cells, a total number of helper T cells and a total number of killer T cells are calculated.

Preferably, in the immune cell subgroup detection part 3, an installed high-speed detection program can detect immune cell subgroup information based on at least the above predetermined white blood cell information and its surface antigen CD number analysis information.

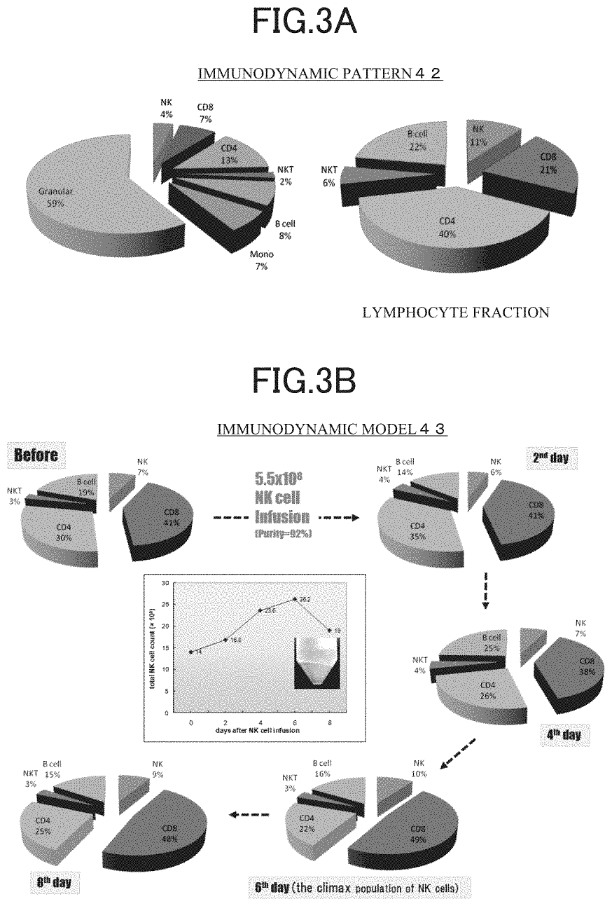

The immunodynamic change analyzing part 4 has a function of generating an immunodynamic pattern 42 as shown in FIG. 3A or an immunodynamic model 43 as shown in FIG. 3B based on information detected or obtained by the above basic information generating part 2 and immune cell subgroup detection part 3 (hereinafter referred to as immune cell basic data). The immune cell basic data includes at least the number of white blood cells, and the numbers and proportions of basophils, eosinophils, neutrophils, lymphocytes, monocytes, CD3 antigens, CD56 antigens (and/or CD16 antigens), CD4 antigens and CD8 antigens in white blood cells.

Based on the above immune cell basic data of the subject, the immunodynamic change analyzing part 4 identifies the immunodynamic pattern of the subject and maps the relation between the subject and the standard value based on the immune cell basic data.

The immunodynamic change analyzing part 4 adaptively provides a prompt for collecting additional data based on the immunodynamic pattern 42 of the subject in consideration of health conditions (pathological conditions). The prompt is generated based on the immunodynamic model 43.

That is, the immunodynamic change analyzing part 4 analyzes immunodynamics of the subject more quickly and accurately based on the increase or decrease in the number of white blood cells, granulocytes (eosinophils, basophils, and neutrophils), monocytes, and lymphocytes (helper T cells, killer T cells, B cells, NK cells, and NKT cells), proportions thereof and the like, and displays the analysis result on the display part 6.

CD3 is an antigen on the membrane surface of all T cells. CD4 is an antigen on the membrane surface of helper T cells (CD4 positive T cells). CD8 is an antigen on the membrane surface of killer T cells (CD8 positive T cells). CD16 is an antigen on the membrane surface of NK cells. CD56 is an antigen on the membrane surface of NK cells and NKT cells.

Hereinafter, positive may be represented as “+” and negative may be represented as “−.” Helper T cells are cells that express a characteristic of “CD3+CD4+,” killer T cells are cells that express a characteristic of “CD3+CD8+,” B cells are cells that express a characteristic of “CD3-CD56-,” NK cells are cells that express a characteristic of “CD3-CD56+,” and NKT cells are cells that express a characteristic of “CD3+CD56+.”

The CD number analysis result receiver receives at least information about CD3 antigens, CD56 antigens (and/or CD16 antigens), CD4 antigens and CD8 antigens.

Hereinafter, an example of operations of the multi-parameter automatic blood cell counting device 1 according to the present embodiment will be described with reference to the following Example 1.

### Example 1

In a first process, blood is collected from the subject, and the basic information generating part 2 analyzes the blood, acquires information about the number of white blood cells, and a proportion (%) of basophils, a proportion (%) of eosinophils, a proportion (%) of neutrophils, a proportion (%) of lymphocytes, and a proportion (%) of monocytes in white blood cells, and CD classification analysis information about CD3 antigens, CD56 antigens (and/or CD16 antigens), CD4 antigens and CD8 antigens and stores the information in the storage part 5.

In a second process, the immune cell subgroup detection part 3 calculates a total number of white blood cells (immune cells) in the body of the subject and a total number (number) of each of white blood cell subgroups (immune cell subgroups) based on the number of white blood cells and white blood cell fraction information acquired by the basic information generating part 2.

Specifically, the total number of white blood cells (immune cells) and the white blood cell subgroups (immune cell subgroups: granulocytes (eosinophils, basophils, and neutrophils), monocytes, lymphocytes (helper T cells, killer T cells, B cells, NK cells, and NKT cells)) are detected (calculated) using a high-speed calculation program bundled in the immune cell subgroup detection part 3. The high-speed calculation program includes at least the following Calculation Formulae (1) to (5). Here, the total number of white blood cells is calculated by Calculation Formula (1) when the subject is male and calculated by Calculation Formula (2) when the subject is female.

(Math. 1)

The total number of white blood cells=the number of white blood cells (/μl)×5,000 (blood volume) ml×1,000 (1,000 μl=1 ml) (1)

The total number of white blood cells=the number of white blood cells (/μl)×4,500 (blood volume) ml×1,000 (1,000 μl=1 ml) (2)

The total number of granulocytes=the total number of white blood cells×granulocytes (basophils+eosinophils+neutrophils) (%) (3)

The total number of monocytes=the total number of white blood cells×monocytes (%) (4)

The total number of lymphocytes=the total number of white blood cells×lymphocytes (%) (5)

Subsequently, the immune cell subgroup detection part 3 detects a total number of T cells in the lymphocyte subgroups based on the detected total number of lymphocytes and the CD classification analysis information acquired by the basic information generating part 2.

Specifically, the total number of T cells in the lymphocyte subgroups is detected (calculated) using a high-speed calculation program bundled in the immune cell subgroup detection part 3 including the following Calculation Formula (6).

Subsequently, the immune cell subgroup detection part 3 detects a total number of helper T cells and a total number of killer T cells based on the calculated total number of T cells and the CD classification analysis information acquired by the basic information generating part 2.

The total number of helper T cells and the total number of killer T cells are detected (calculated) using a high-speed calculation program bundled in the immune cell subgroup detection part 3 including the following Calculation Formulae (7) and (8).

Subsequently, the immune cell subgroup detection part 3 detects a total number of non-T cells other than T cells based on the detected total number of T cells and total number of lymphocytes and the CD classification analysis information acquired by the basic information generating part 2.

The total number of non-T cells is detected (calculated) using a high-speed calculation program bundled in the immune cell subgroup detection part 3 including the following Calculation Formula (9).

Subsequently, the immune cell subgroup detection part 3 detects a total number of B cells, a total number of NK cells and a total number of NKT cells based on the detected total number of non-T cells and total number of T cells and the CD classification analysis information acquired by the basic information generating part 2.

The total number of B cells, the total number of NK cells and the total number of NKT cells are detected (calculated) using a high-speed calculation program bundled in the immune cell subgroup detection part 3 including the following Calculation Formulae (10), (11), and (12).

(Math. 2)

The total number of T cells=the total number of lymphocytes×CD3(+) (%) (6)

The total number of CD4(+) T cells=the total number of T cells×CD4(+) (%) (7)

The total number of CD8(+) T cells=the total number of T cells×CD8(+) (%) (8)

The total number of non-T cells=the total number of lymphocytes×(100−CD3(+)) (9)

The total number of B cells=the total number of non-T cells×(CD16(−)/CD56(−)) (%) (10)

The total number of NK cells=the total number of non-T cells×((CD16(+)/CD56(+))+(CD16(+)/CD56(−))) (%) (11)

The total number of NKT cells=the total number of T cells×((CD16(−)/CD56(+)) (%) (12)

Here, according to Calculation Formulae (1) to (12), the number of cells in 1 μl of each component can be calculated. The immune cell subgroup detection part 3 stores the calculated result data in the storage part 5.

In a third process, the immunodynamic change analyzing part 4 creates immunodynamic information about the subject based on the data information (immune cell basic data) detected (calculated) by the basic information generating part 2 and the immune cell subgroup detection part 3 and the data stored in the storage part 5. The immunodynamics in this specification are subject information about the immune status of the subject displayed based on the numbers and proportions of white blood cells, basophils, eosinophils, neutrophils, lymphocytes, monocytes, CD3 antigens, CD56 antigens (and/or CD16 antigens), CD4 antigens and CD8 antigens in white blood cells of the subject detected immediately when blood is sampled (within 2 hours after blood sampling).

That is, the immunodynamic change analyzing part 4 analyzes the immune status of the subject quickly and accurately based on the numbers and proportions of white blood cells, granulocytes (eosinophils, basophils, and neutrophils), monocytes, and lymphocytes (helper T cells, killer T cells, B cells, NK cells, and NKT cells) detected by the above basic information generating part 2 and immune cell subgroup detection part 3, forms a pattern of the immune status, and displays immunodynamics of the subject. The immunodynamic change analyzing part 4 is composed of hardware (circuits, dedicated logics, and the like), software (such as that operating in a general-purpose computer system or a dedicated machine), and like.

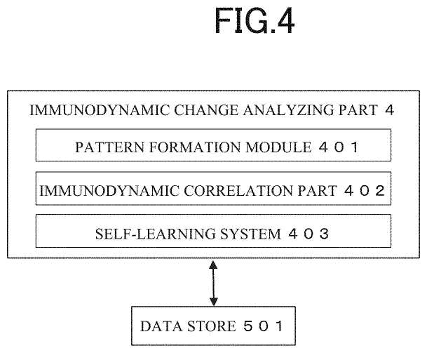

As shown in FIG. 4, the immunodynamic change analyzing part 4 specifically includes a pattern formation module 401, an immunodynamic correlation part 402, and a self-learning system 403. The immunodynamic change analyzing part 4 is connected to one or a plurality of data stores 501 or (and) the storage part 5 for storing data (for example, information, models, feedbacks, subject profiles, and the like).

The pattern formation module 401 is image formation software for forming a pattern of the immune status, partitions the immune status, and forms an immune status pattern (for example, an immunodynamic pattern). The self-learning system 403 has a self-learning function of learning according to blood count data and characterizes a correlation between components of blood counts.

### Pattern Formation Example 1

In Pattern Formation Example 1, functions of the immunodynamic change analyzing part 4 will be described based on pattern formation related to influenza infection.

In Pattern Formation Example 1, first, the self-learning system 403 extracts cell data correlated with influenza virus based on the total number of white blood cells (immune cells) and the white blood cell subgroups (immune cell subgroups) of a certain number of influenza infected persons (hereinafter referred to as infected persons) selected at random, which are stored in the storage part 5 (or the data store 501), and stores the data in the storage part 5 (or the data store 501). Here, the cell data extracted by the self-learning system 403 includes the numbers of total granulocytes, total lymphoid cells, CD4 antigens, CD8 antigens, NK cells, NKT cells and B cells shown in Table 1.

TABLE 1

| | 21-year | 37-year | 31-year | 47 -year | 32-year | 35-year | 48-year | 25-year | 48-year | 65-year |
|---|---|---|---|---|---|---|---|---|---|---|
| | male | male | male | female | male | male | female | female | male | male |
| Total | 4401 | 4073 | 4068 | 2026 | 2545 | 2347 | 1908 | 2754 | 2850 | 7250 |
| granulocytes | | | | | | | | | | |
| Total | 377 | 631 | 291 | 169 | 269 | 274 | 581 | 325 | 351 | 921 |
| lymphoid cells | | | | | | | | | | |
| CD4 | 79 | 82 | 54 | 40 | 40 | 45 | 108 | 55 | 46 | 97 |
| CD8 | 97 | 64 | 40 | 33 | 84 | 57 | 97 | 33 | 40 | 178 |
| NK | 31 | 136 | 27 | 13 | 43 | 56 | 87 | 23 | 29 | 146 |
| NKT | 18 | 14 | 5 | 6.6 | 15 | 15 | 10 | 6 | 10 | 27 |
| B | 90 | 202 | 110 | 45 | 46 | 78 | 166 | 123 | 121 | 268 |

The self-learning system 403 determines a pattern range (influenza infection pattern and pseudo-influenza infection pattern) indicating a characteristic of influenza infection based on the numbers of total granulocytes, total lymphoid cells, CD4 antigens, CD8 antigens, NK cells, NKT cells and B cells stored in the storage part 5 (or the data store 501), and stores the range in the storage part 5 (or the data store 501). The data in Table 1 is calculated by the immune cell subgroup detection part 3 based on the number of white blood cells and the white blood cell fraction information of infected persons acquired by the basic information generating part 2 and corresponds to infected immune cell basic data of the infected persons extracted by the self-learning system 403.

The self-learning system 403 calculates a total number of the number of CD4 antigens, the number of CD8 antigens, the number of NK cells, the number of NKT cells and the number of B cells of the infected persons based on the infected immune cell basic data of the infected persons shown in Table 1, calculates proportions of the number of CD4 antigens, the number of CD8 antigens, the number of NK cells, the number of NKT cells and the number of B cells shown in the following Table 2, and stores the results in the storage part 5 (or the data store 501).

TABLE 2

| | 21-year | 37-year | 31-year | 47-year | 32-year | 35-year | 48-year | 25-year | 48-year | 65-year | Ave. |
|---|---|---|---|---|---|---|---|---|---|---|---|
| | male | male | male | female | male | male | female | female | male | male | value |
| CD4 | 25% | 16% | 23% | 29% | 18% | 18% | 23% | 23% | 19% | 14% | 21% |
| CD8 | 31% | 13% | 17% | 24% | 37% | 23% | 21% | 14% | 16% | 25% | 22% |
| NK | 10% | 27% | 11% | 9% | 19% | 22% | 19% | 10% | 12% | 20% | 16% |
| MKT | 6% | 3% | 2% | 5% | 7% | 6% | 2% | 2% | 4% | 4% | 4% |
| B | 28% | 41% | 47% | 33% | 20% | 31% | 35% | 51% | 49% | 37% | 37% |
| CD4/8 | 0.88 | 0.41 | 0.49 | 0.89 | 0.87 | 0.58 | 0.65 | 0.45 | 0.38 | 0.36 | 0.59 |
| Total | 0.09 | 0.15 | 0.07 | 0.08 | 0.11 | 0.12 | 0.3 | 0.12 | 0.12 | 0.13 | 0.13 |
| lymphoid | | | | | | | | | | | |
| cells/total | | | | | | | | | | | |
| granules | | | | | | | | | | | |
| Body | 39.4 | 38.6 | 38.8 | 38.2 | 40 | 38.8 | 38.8 | 39 | 38.5 | 38.4 | 38.8 |
| temperature | | | | | | | | | | | |
| (° C.) | | | | | | | | | | | |
| Pulse rate | 97 | 122 | 100 | 88 | 118 | 125 | 115 | 102 | 110 | 130 | 111 |

In addition, the self-learning system 403 simultaneously shows reference data determined to be related to the characteristic of influenza infection, for example, the body temperature and pulse rate as shown in Table 2, when the pattern range indicating a characteristic of influenza infection is determined.

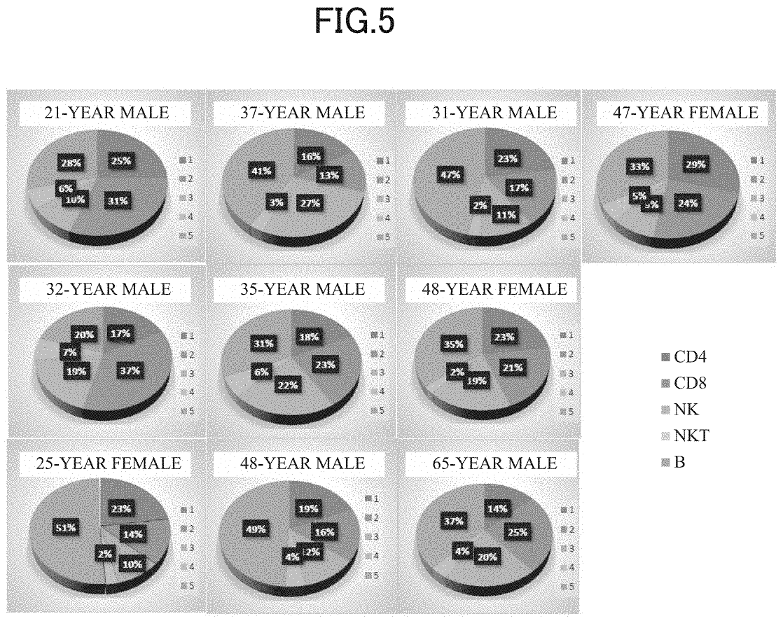

Next, the pattern formation module 401 forms an image of the proportions of the number of CD4 antigens, the number of CD8 antigens, the number of NK cells, the number of NKT cells and the number of B cells of the infected persons shown in Table 2, which are stored in the storage part 5 (or the data store 501), and displays the image on the display part 6. FIG. 5 is a diagram showing proportions of the number of CD4 antigens, the number of CD8 antigens, the number of NK cells, the number of NKT cells and the number of B cells of infected persons displayed on the display part 6.

In addition, the self-learning system 403 calculates a ratio (CD4/B) of the number of CD4 antigens to the number of B antigens, and a ratio of total lymphoid cells to total granules (total lymphoid cells/total granules) of each infected person and stores the results in the storage part 5 (or the data store 501). In addition, the self-learning system 403 calculates respective average values (referred to as comparison influenza infection pattern standard data) of proportions of the number of CD4 antigens, proportions of the number of CD8 antigens, proportions of the number of NK cells, proportions of the number of NKT cells and proportions of the number of B cells of the infected persons shown in Table 2 and stores the results in the storage part 5 (or the data store 501).

The pattern formation module 401 can display information for determining whether the subject is infected with influenza on the display part 6 using the comparison influenza infection pattern standard data stored in the storage part 5.

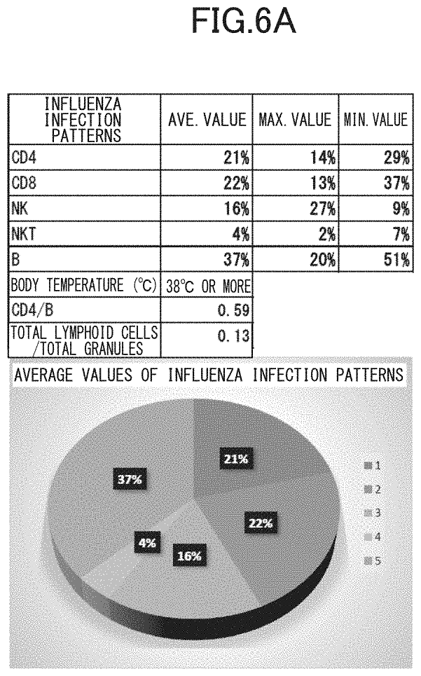

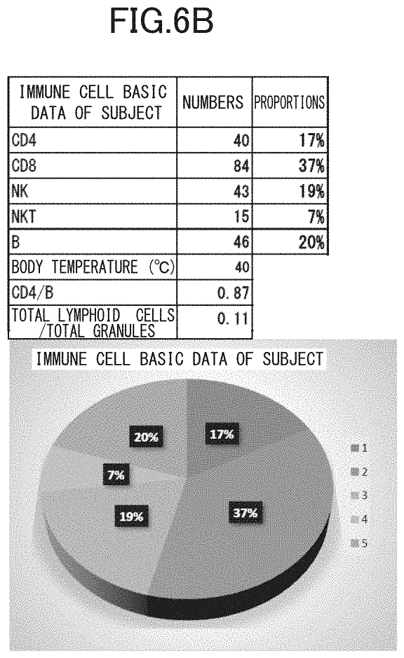

That is, in the multi-parameter automatic blood cell counting device 1 according to the present embodiment, the self-learning system 403 compares immune cell basic data of the subject with various types of data stored in the storage part 5, selects immune cell data (hereinafter referred to as comparison immune cell data) close to the immune cell basic data of the subject, and displays the results on the display part 6 (refer to FIG. 6A and FIG. 6B). Therefore, the staff can easily determine whether the subject is infected with influenza virus with reference to the immune cell basic data of the subject and the comparison immune cell data displayed on the display part 6, and can prevent the spread of infection at an early stage.

Hereinafter, a function of displaying infectious disease determination information of the multi-parameter automatic blood cell counting device 1 according to the present embodiment will be described using clinical cases of actual influenza infection patterns.

### Determination Example 1

First, in the medical field in which the multi-parameter automatic blood cell counting device 1 according to the present embodiment is provided, blood of a subject is collected, and using the basic information generating part 2, the number of white blood cells of the subject, the white blood cell image (white blood cell fraction information), and the analysis result of a surface antigen CD number of white blood cells (immune cells) are acquired, and the results are stored in the storage part 5.

Next, the immune cell subgroup detection part 3 calculates a total number of white blood cells (immune cells) in the body of the subject and a total number (number) of each of white blood cell subgroups (immune cell subgroups) based on the number of white blood cells and white blood cell fraction information acquired by the basic information generating part 2, and stores the results in the storage part 5.

The pattern formation module 401 forms an image of the numbers and proportions of total granulocytes, total lymphoid cells, CD4 antigens, CD8 antigens, NK cells, NKT cells and B cells (hereinafter referred to as immune cell basic data of the subject) from the total number of white blood cells (immune cells) and the white blood cell subgroups (immune cell subgroups) of the subject and displays the image on the display part 6.

Subsequently, the pattern formation module 401 instructs the self-learning system 403 to select comparison immune cell data similar to the immune cell basic data of the subject from the immune cell data group stored in the storage part 5 (or the data store 501) (selection operation).

The self-learning system 403 reports the fact that the selection operation is completed to the pattern formation module 401 when the self-learning system 403 determines and selects comparison immune cell data similar to the immune cell basic data of the subject from the immune cell data group stored in the storage part 5 (or the data store 501). Here, the comparison immune cell data selected by the self-learning system 403 is comparison influenza infection pattern standard data.

After the report indicating that the selection operation is completed is received from the self-learning system 403, the pattern formation module 401 displays comparison influenza infection pattern standard data on the display part 6.

As a result, the pattern formation module 401 displays simultaneously the immune cell basic data of the subject and the comparison influenza infection pattern standard data on the display part 6. In addition, when it is determined that the immune cell basic data of the subject is within the range of comparison influenza infection pattern standard data, the pattern formation module 401 displays the influenza infection pattern (positive). When it is determined that the immune cell basic data of the subject is not within the range of comparison influenza infection pattern standard data, the pattern formation module 401 displays that it is not an influenza infection pattern (negative).

FIG. 6A is a diagram showing immune cell basic data of the subject that is displayed on the display part 6 by the pattern formation module 401. FIG. 6B is a diagram showing comparison influenza infection pattern standard data that is displayed on the display part 6 by the pattern formation module 401.

A medical staff can determine the infection status of the subject simply and quickly with reference to the immune cell basic data of the subject and the comparison influenza infection pattern standard data as shown in FIG. 6A and FIG. 6B, and can detect infected persons at an early stage and effectively prevent the spread of infection.

The pattern formation module 401 in the present embodiment is not limited to that of display of the immune cell basic data of the subject and the comparison immune cell data. The pattern formation module 401 in the present embodiment has a function of calculating proportions of the number of NK cells, the number of helper T cells, the number of killer T cells, the number of B cells and the number of NKT cells of the subject, forming a graph, and forming pattern classification.

In the pattern classification, for example, based on the numbers and proportions of cells in respective components of the white blood cell subgroups, the numbers and proportions of cells of monocytes and the lymphocyte subgroups, and the number and proportion of cells of only the lymphocyte subgroups, the immunodynamic (normal or abnormal) pattern of the immune status is identified.

Hereinafter, an operation of the pattern formation module 401 forming pattern classification will be described using an output example (normal immune status pattern I) of the immune status of healthy persons with no disease (hereinafter referred to as “healthy persons”), an output example (abnormal condition I) of the immune status of patients with gastric cancer, an output example (abnormal condition II) of patients with rheumatoid arthritis, and an output example (abnormal condition III) of the immune status with patients with atopic dermatitis.

### Output Example 1

First, blood of patients with gastric cancer (subject) is collected, and the basic information generating part 2 and the immune cell subgroup detection part 3 acquire immune cell basic data of patients with gastric cancer.

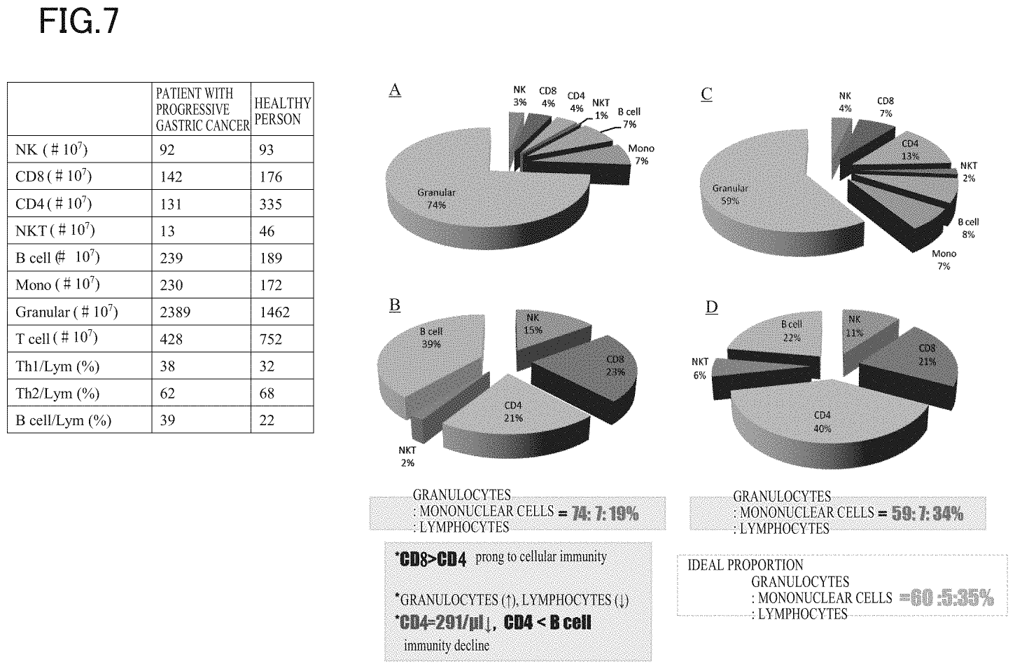

Next, the pattern formation module 401 generates pie charts of the immune status of patients with gastric cancer as shown in (A) and (B) in FIG. 7 based on the immune cell basic data of patients with gastric cancer and outputs the results to the display part 6. The multi-parameter automatic blood cell counting device 1 according to the present embodiment can immediately display the immune status of patients with gastric cancer.

In order to check the immune status of patients with gastric cancer, the pattern formation module 401 displays comparison information with the immune status of healthy persons. Specifically, the pattern formation module 401 acquires data of the immune status of healthy persons suitable for comparison of the patients with gastric cancer from the storage part 5, generates pie charts of the immune status of healthy persons as shown in (C) and (D) in FIG. 7, and displays the results on the display part 6. In this output example, the pattern formation module 401 simultaneously displays the pie charts of the immune status of healthy persons as shown in (C) and (D) in FIG. 7 and the pie charts of the immune status of patients with gastric cancer as shown in (A) and (B) in FIG. 7.

(A) in FIG. 7 is a pie chart showing proportions of components of the white blood cell subgroups of patients with gastric cancer. (B) in FIG. 7 is a pie chart showing proportions of only the lymphocyte subgroups of patients with gastric cancer. (C) in FIG. 7 is a pie chart showing proportions of components of the white blood cell subgroups of healthy persons. (D) in FIG. 7 is a pie chart showing the proportion of only the lymphocyte subgroups of healthy persons.

As shown in (A) and (B) in FIG. 7, the number of CD4 cells of patients with gastric cancer is 1.31 billion, granulocytes:mononuclear cells:lymphocytes=74:7:19%, and sympathetic nerve dominance and granulocyte-proliferative lymphocyte decrease are shown. The number of cells of the immune phase central lymphocyte subgroups 262 cells/μl (<300 cells/μl), and the proportion value of CD4 is smaller than the proportion value of B cells and the proportion value of CD8, and suggests immune-decline. In addition, since the Th1 cell-mediated immune group proportion (NK group+CD8 group)>Th2 humoral immune group proportion (NKT group+B group), the cell-mediated immparty tends to dominant. In this case, the pattern formation module 401 identifies the immune status of the patients with gastric cancer as an “immune-decline pattern.” In addition, the pattern formation module 401 forms a pattern based on the following immparty evaluation reference data A) to C).

A) Self-immunity Healthy-degree (health level of
self-immunity)
Number of cells of CD4 Helper-T cell subgroups
(= helper T cells immunity center)/μL/ml
CD4 > 600/μl → normal Immunity (normal)
CD4 600 to 400/μl → Immune-decline
CD4 400 to 200/μl → Immune-impaired
CD4 < 200/μl → Immune-crisis
B) Body direction of immunity
Th1 cell-mediated immunity = NK + CD8 (killer-T)
Th2 humoral immunity = NKT + B cell
CD4 > CD8 > B cell direct proportion (≈lymphatic
subgroup proportional pattern)
C) Body immune autonomic nerve balance
Granulocytes:mononuclear cells:lymphocytes =
60:5:35% (average value for normal persons) →
sympathetic nerves (granulocytes > lymphocytes %)/
parasympathetic nerve dominance (lymphocytes >
granulocytes %)
D) Compensatory change in body immune cell group
(based on monitoring data from the begging because
it constantly changes)
Other patterns that change

In this example, the immparty evaluation reference data A) to C) are stored in the data store 501 (or the storage part 5) in advance.

On the other hand, as shown in (C) and (D) in FIG. 7, the number of CD4 cells of healthy persons is 3.35 billion, and granulocytes:mononuclear cells:lymphocytes=59:7:34%. The average value for normal persons is granulocytes:mononuclear cells:lymphocytes=60:5:35%.

In this case, the multi-parameter automatic blood cell counting device 1 of the present embodiment shows that the immune status of the patients with gastric cancer is an “immune-decline pattern,” and displays a characteristic value indicating a decline in immparty (for example, the numerical value on the left side in FIG. 7) in comparison with the above immune status of the healthy persons, and stores the results in the storage part 5.

Therefore, the medical staff can check the immune status of the examinee in a timely manner, acquire the dynamic state of immparty of the examinee, and accurately determine the immune status using the multi-parameter automatic blood cell counting device 1 of the present embodiment. In addition, the multi-parameter automatic blood cell counting device 1 of Example 1 can be composed of hardware (circuits, dedicated logics, and the like) and software (such as that operating in a general-purpose computer system or a dedicated machine) required for analyzing the number of white blood cells, the white blood cell image (white blood cell fraction information), and the surface antigen CD number of certain white blood cells (immune cells). As a result, the multi-parameter automatic blood cell counting device 1 of the present embodiment is made compact. In addition, data related to the immune status acquired by at least a combination of a large number of devices such as a blood cell count detection device, a flow cytometer and a PCR in the related art can be acquired immediately using only the multi-parameter automatic blood cell counting device 1 of the present embodiment.

In addition, when the medical staff uses the multi-parameter automatic blood cell counting device 1 of the present embodiment, it is possible to visually recognize data about immunodynamics of the examinee and the pattern of the immune status, and perform identification quickly and accurately, and shorten the examination time without performing each separate calculation as shown in JP2008-89382A in order to acquire immunodynamics.

### Output Example 2

In Output Example 2, an example in which the immune status belonging to the Th2 humoral immune dominant tendency pattern is output is shown. In consideration of characteristics of the Th2 humoral immune dominant tendency pattern, an example in which subjects are patients with rheumatoid arthritis and patients with atopic dermatitis is described.

As in Output Example 1, first, blood of the subject is collected, and the basic information generating part 2 and the immune cell subgroup detection part 3 acquire immune cell basic data 41 of the subject.

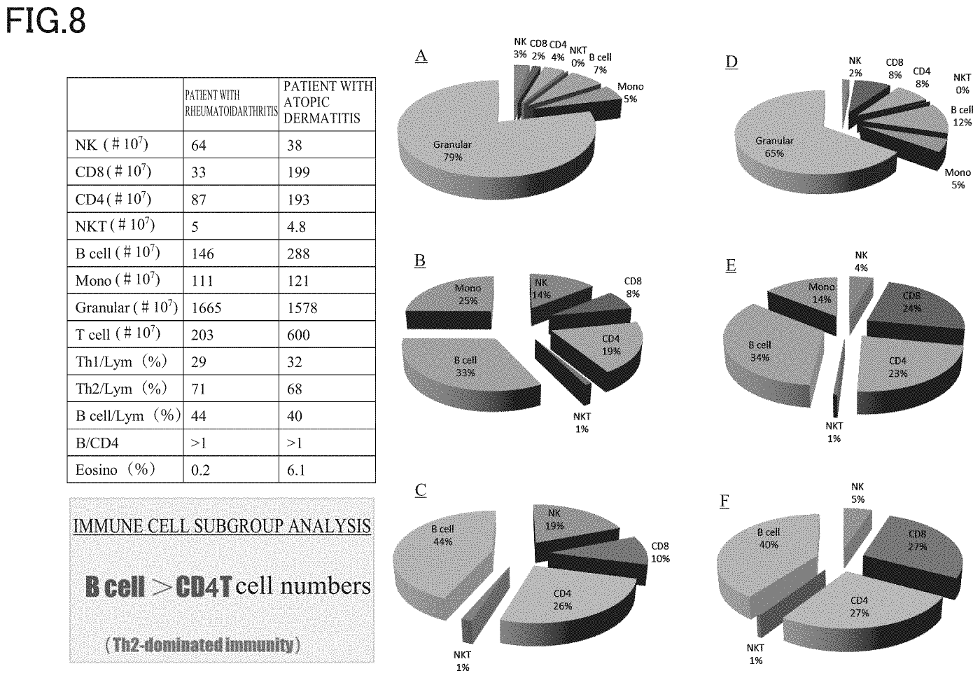

Next, the pattern formation module 401 generates pie charts of the immune status of patients with rheumatoid arthritis and the immune status of patients with atopic dermatitis as shown in FIG. 8 based on the immune cell basic data 41 of the subjects. (A) in FIG. 8 is a pie chart showing proportions of components of the white blood cell subgroups of patients with rheumatoid arthritis. (B) in FIG. 8 is a pie chart showing proportions of the lymphocyte subgroups and single cells of patients with rheumatoid arthritis. (C) in FIG. 8 is a pie chart showing the proportion of only the lymphocyte subgroups of patients with rheumatoid arthritis. (D) in FIG. 8 is a pie chart showing proportions of components of the white blood cell subgroups of patients with atopic dermatitis. (E) in FIG. 8 is a pie chart showing proportions of the lymphocyte subgroups and single cells of patients with atopic dermatitis. (F) in FIG. 8 is a pie chart showing the proportion of only the lymphocyte subgroups of patients with atopic dermatitis.

As shown in (A) and (C) in FIG. 8, the number of CD4 cells of an 82-year-old female patient with rheumatoid arthritis is 870 million, the number of CD8 cells is 330 million, and granulocytes:mononuclear cells:lymphocytes=79:5:16%. The proportion value of CD4 of patients with rheumatoid arthritis is smaller than the proportion value of B cells (inverse proportional phenomenon of the lymphocyte subgroups) but is larger than the proportion value of CD8, and since the Th2 humoral immune group proportion (NKT group+B group)>Th1 cell-mediated immparty (NK group+CD8 group), the Th2 humoral immune dominant tendency pattern is shown. In this case, the multi-parameter automatic blood cell counting device 1 of the present embodiment determines that the immune status of the patients with rheumatoid arthritis is a “Th2 humoral immune dominant tendency pattern” and displays a characteristic of the immune status in comparison with the immune status of healthy persons.

As shown in (D) and (F) in FIG. 8, the number of CD4 cells of patients with atopic dermatitis is 1.93 billion, the number of CD8 cells is 1.99 billion, and granulocytes:mononuclear cells:lymphocytes=65:5:30%. In addition, the proportion value of CD4 of patients with atopic dermatitis is smaller than the proportion value of B cells (inverse proportional phenomenon of the lymphocyte subgroups), and is almost equal to the proportion value of CD8. As in the above patients with chronic joints, since the Th2 humoral immune group proportion (NKT group+B group)>Th1 cell-mediated immparty (NK group+CD8 group), the Th2 humoral immune dominant tendency pattern is shown. In this case, the multi-parameter automatic blood cell counting device 1 of the present embodiment shows that the immune status of the patients with atopic dermatitis is a “Th2 humoral immune dominant tendency pattern” and displays a characteristic of the immune status in comparison of the immune status of healthy persons.

### Output Example 3

In Output Example 3, an example in which, in order to accurately determine the immune status and pathological conditions and in order to compare with other pathological condition patterns, a pathological condition pattern (comparison state pattern) different from the immune status pattern of the subject is simultaneously displayed for comparison is described. Since the immune cell basic data 41 of the subject is acquired by the same method as in Output Example 1, descriptions thereof will be omitted.

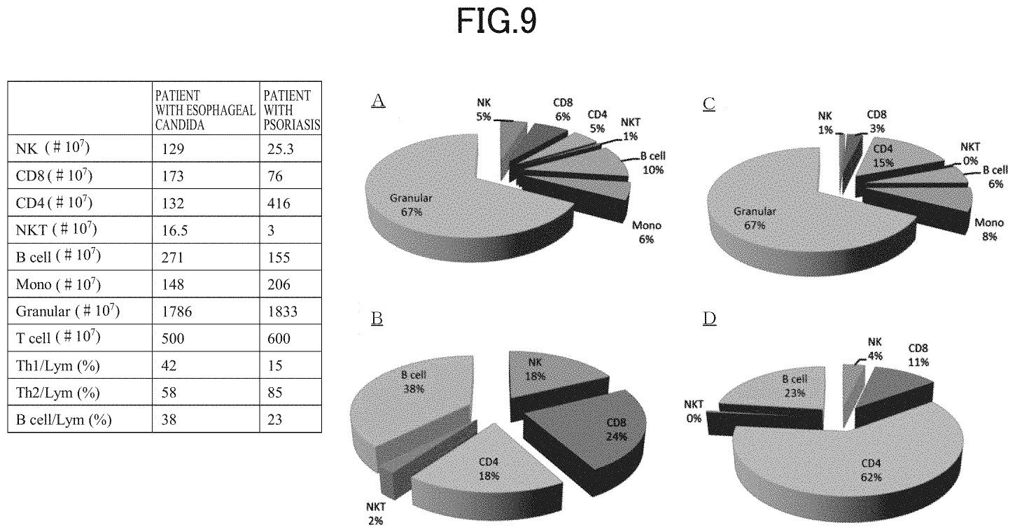

The pattern formation module 401 generates pie charts of the immune status of patients with esophageal candida and the immune status of patients with psoriasis as shown in FIG. 9 based on the immune cell basic data 41 of the subjects. (A) in FIG. 9 is a pie chart showing proportions of components of the white blood cell subgroups of patients with esophageal candida. (B) in FIG. 9 is a pie chart showing the proportion of only the lymphocyte subgroups of patients with esophageal candida. (C) in FIG. 9 is a pie chart showing proportions of components of the white blood cell subgroups of patients with psoriasis. (D) in FIG. 9 is a pie chart showing the proportion of only the lymphocyte subgroups of patients with psoriasis.

As shown in (A) and (B) in FIG. 9, the number of CD4 cells of patients with esophageal candida is 1.32 billion, the number of CD8 cells is 1.73 billion, and granulocytes:mononuclear cells:lymphocytes=67:6:27%. In addition, the number of CD4 cells of patients with esophageal candida indicates the immune-decline state of 264 cells/μl (<400 cells/μl), and the proportion value is smaller than the proportion value of B cells and the proportion value of CD8, and suggests the inverse proportional phenomenon of the lymphocyte subgroups. Since the Th1 cell-mediated immune group proportion (NK group+CD8 group)>Th2 humoral immparty (NKT group+B group), the cell-mediated immparty dominant tendency is shown. In this case, the multi-parameter automatic blood cell counting device 1 of the present embodiment shows that the immune status of the patients with esophageal candida is a cell-mediated immparty dominant tendency pattern, and displays a characteristic of the immune status in comparison with the immune status of healthy persons.

On the other hand, the immune status of patients with psoriasis shown in (C) and (D) in FIG. 9 has a value that is significantly different from that of the immune status of patients with esophageal candida. Granulocytes:mononuclear cells:lymphocytes of patients with psoriasis=67:8:25%, which are values almost the same as those of the above patients with esophageal candida, and CD4 of patients with psoriasis indicates an immunostimulatory state with 4.16 billion and 832 cells/μl (>600 cells/μl), which is larger than 760 million for CD8. In addition, the proportion value of CD4 of patients with psoriasis is much larger than the proportion value of B cells and the proportion value of CD8, and the proportion of NKT cells is almost zero, which suggests a possibility of the Th17 cell-mediated immparty dominant tendency rather than the Th2 humoral immune dominant tendency pattern. In this case, the multi-parameter automatic blood cell counting device 1 of the present embodiment suggests that the immune status of the patients with psoriasis is the “Th17 cell-mediated immparty dominant tendency pattern,” and displays a characteristic of the immune status in comparison of the immune status of healthy persons.

The comparison state pattern in Output Example 3 is provided by the self-learning system 403 of this specification.

The self-learning system 403 can select one or a plurality of comparison state patterns having the best correlation with the immune status of the subject by statistical calculation (mathematical optimization calculation and the like) or selection by a clinical staff based on the data stored in the data store 501. For example, numerical analysis software can be used for mathematical optimization calculation. For example, general numerical analysis software SciPy can be used.

For example, the self-learning system 403 is connected to at least the data store 501 in which immunodynamic analysis reference data is stored. The immunodynamic analysis reference data is only an example as reference information for analyzing and defining the change in immunodynamics, and is beneficial information for clinical staff when correlation statistical data of the change in immunodynamics is analyzed and defined.

Reference information data for analyzing and defining the change in immunodynamics may be information supplemented in a timely manner based on information about medical clinical fields and information about specialized magazines.

In addition, the clinical staff can additionally supplement the reference information data for analyzing and defining the change in immunodynamics to those specialized in the beneficial specialty therapeutic field based on their own experience.

Regarding statistical calculation of the self-learning system 403, if a comparison state pattern having the best correlation with the immune status of the subject can be selected, the following simple calculation method may be used for selection.

In Calculation Example 1, multiple regression analysis is performed using the number of NK cells as a dependent variable, and the numbers of helper T cells, killer T cells, B cells, and NKT cells as independent variables.

The partial regression coefficients of the numbers of helper T cells, killer T cells, B cells, and NKT cells are obtained and a significance difference test is performed and the results are displayed.

Next, a multiple correlation coefficient is obtained, a significance difference test is performed, and the results are displayed.

Contribution rates of the numbers of helper T cells, killer T cells, B cells, and NKT cells are obtained and displayed.

The correlation between the number of NK cells and the numbers of helper T cells, killer T cells, B cells, and NKT cells is obtained and displayed.

Regarding an example of correlation involved in activation of cell-mediated immparty, a total sum of NK cells and killer T cells and the increase or decrease thereof, and the like are analyzed, the Th1 cell-mediated immune status in which Th1 cells are involved is compared with the immune status of the subject, and the most suitable one is selected from the statistical data group. Th1 cells mainly produce IFN-γ and are involved in activation of cell-mediated immparty and the like.

In Calculation Example 2, based on the correlation between the numbers of NKT cells and NK cells, helper T cells, killer T cells, and B cells, accurate correlation information about the humoral immune status and the like is obtained, and the most suitable one is selected from the statistical data group in comparison with the immune status of the subject.

The multiple regression analysis of Calculation Example 2 is performed by the same method as in the above Calculation Example 1. Then, the correlation between the numbers of NKT cells, NK cells, helper T cells, killer T cells, and B cells is obtained, and the most suitable one is selected from the statistical data group in comparison with the immune status of the subject.

Regarding an example of a correlation involved in the humoral immune status, a total sum of NKT cells and B cells and the increase or decrease thereof, and the like are analyzed, and thus it is possible to ascertain the Th2 humoral immune status in which Th2 cells are involved. Th2 cells mainly produce interleukin-4 (IL-4) and are involved in humoral immparty.

The above Calculation Example 1 and Calculation Example 2 are only examples in which a correlation for analyzing the change in immunodynamics is derived. The multi-parameter automatic blood cell counting device of the present embodiment includes those showing significant numerical values indicating immunodynamics based on optimization calculation and the like in addition to the correlation method indicating immunodynamics in the above Calculation Example 1 and Calculation Example 2. That is, those that provide a comparison state pattern for statistical analysis of the correlation and change in immunodynamics of the immune cell subgroups acquired based on the information obtained using the immune cell basic data 41 of the subject and can be immediately applied to the immune status of the subject can be used for the multi-parameter automatic blood cell counting device of the present embodiment. In addition, according to joint analysis of analysis data of the multi-parameter automatic blood cell counting device of the present embodiment and measurement data in other fields, application to forming an image of new information analysis is possible. For example, according to joint analysis of analysis data of the multi-parameter automatic blood cell counting device of the present embodiment and bacterial flora analysis data in intestinal feces, it is possible to help investigate the Th17 mucosal immparty field.

Next, operations of the immunodynamic correlation part 402 will be described based on Output Example 4.

The immunodynamic correlation part 402 displays information about the immunodynamic correlation of the subject with respect to the immune status pattern of the subject formed by the pattern formation module 401. That is, according to administration of related cells, the change in the immune status (change in immunodynamics) of the subject that is sequentially imaged is displayed and a tendency change amount and a reference required dose are displayed.

### Output Example 4

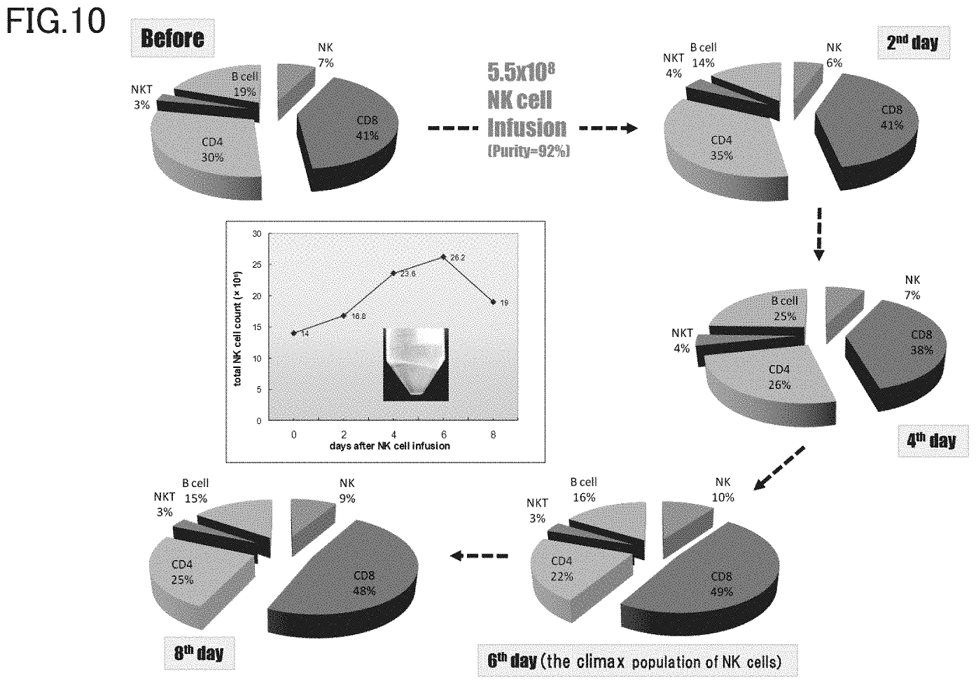

In Output Example 4, NK cells (self-cultured cells) are administered to a subject (a subject 1) having an NK cell decrease pattern, the change (change in immunodynamics) in the immune status that is sequentially imaged is displayed, and the immunodynamic model 43 is formed. FIG. 10 is a diagram showing the immunodynamic model 43 formed by the immunodynamic correlation part 402 according to the present embodiment.

As shown in FIG. 10, the immunodynamic correlation part 402 images and displays the administration effect of NK cells using a physical method such as infusion or injection. The medical staff and the subject 1 can directly and immediately check the immunodynamics of the subject. In addition, in Output Example 4, NK cells are administered to a subject having an NK cell decrease pattern, and the immunodynamic correlation part 402 forms the immunodynamic model 43 indicating the change in the immune status of the subject for a certain period. Specifically, the results of the following operations are imaged and displayed.

First, blood of the subject is collected before NK cells are injected and the results of analysis (the immune cell basic data 41 before administration) are stored in the storage part 5.

Next, self-cultured NK cells (5.5×10⁸ NK cells) having a purity of 92% are injected into the subject, and blood is then collected again two days later, and analysis is performed using the basic information generating part 2 and the immune cell subgroup detection part 3, and the immune cell basic data 41 after administration is generated.

The pattern formation module 401 immediately identifies each of an NK cell decrease pattern and a first NK cell compensation pattern 1 which are the immunodynamic pattern 42 of the subject based on the immune cell basic data 41 before administration and the immune cell basic data 41 after administration.

The immunodynamic correlation part 402 displays a graph showing the increase or decrease in the number of NK cells in the body of the subject 1 based on the NK cell decrease pattern and the first NK cell compensation pattern 1 of the subject 1.

As shown in FIG. 10, when autologous NK cells are once injected by infusion, amplification of Th1 cell-mediated immparty is observed. In addition, when NK cells are injected by infusion, amplification of NK cells in the body is also directly observed.

That is, when a drug contributing to NK cells is injected, the medical staff can ascertain quickly and easily information about actions and effects and change thereof based on the multi-parameter automatic blood cell counting device 1 of the present embodiment, which results in reducing the burden on the patient, and early detection and early treatment.

In addition, the immunodynamic correlation part 402 forms a graph proportional to the dose of NK cells and an increased amount of NK cells in the body of the subject 1 and calculates and displays the digestibility 1 of the dose 1 of NK cells.

In addition, the immunodynamic correlation part 402 selects the optimal data 1 from the data store 501 using the NK cell decrease pattern 1 and the first NK cell compensation pattern 1 of the subject 1 as samples by using the self-learning system 403. The optimal data 1 is data including information about immune status patterns (the NK cell decrease pattern and the first NK cell compensation pattern, the second NK cell compensation pattern, the third NK cell compensation pattern, the fourth NK cell compensation pattern . . . ) that are the same as or similar to the NK cell decrease pattern 1 and the first NK cell compensation pattern 1.

Then, the immunodynamic correlation part 402 displays the NK cell decrease pattern 1 and the first NK cell compensation pattern 1, the second NK cell compensation pattern 1, the third NK cell compensation pattern 1, and the fourth NK cell compensation pattern 1 of the subject 1 from the optimal data 1. In addition, the immunodynamic correlation part 402 displays the dose and digestibility for the second NK cell compensation pattern 1 of the optimal data 1 and displays an estimated dose 2. The estimated dose 2 is a required dose of NK cells for forming the ideal second NK cell compensation pattern of the subject 1. The estimated dose 2 is obtained by a calculation method of the self-learning system 403 based on the dose 1 and digestibility 1 of the subject and the dose and digestibility of the optimal data 1.

In addition, the immunodynamic correlation part 402 generates and displays an immunodynamic model 1 of the subject 1 based on the NK cell decrease pattern 1 and the first NK cell compensation pattern 1 for the subject 1 and the optimal data 1. As shown in FIG. 10, the immunodynamic model 1 of the subject 1 is a model in which the change in immunodynamics from the NK cell decrease pattern 1 to the fourth NK cell compensation pattern 4 is predicted.

The immunodynamic model 1 of the subject 1 allows the medical staff to immediately and dynamically determine the immune status of the subject 1, and immediately make a decision what measures to take. The immunodynamic model 1 allows the subject 1 to directly and immediately visually check his or her own immunodynamics, and to accurately determine his or her own immparty information.

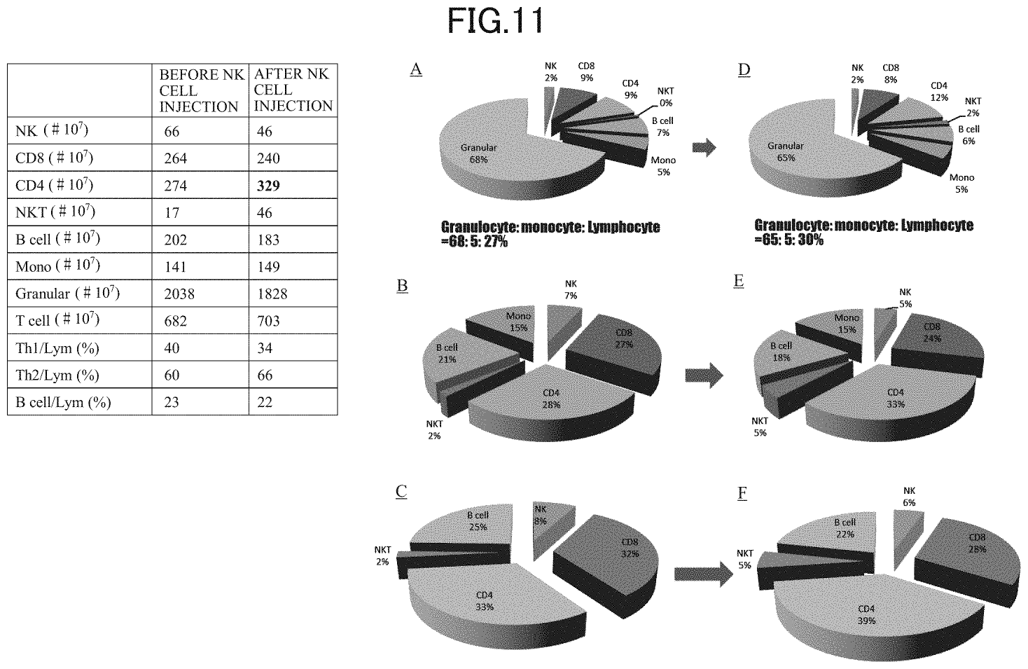

FIG. 11 shows examples in which the change in immunodynamics of NK cells is imaged and displayed for healthy persons.

(A) in FIG. 11 is a pie chart showing proportions of components of the white blood cell subgroups of the subject before NK cells are administered and a calculation example of the number of white blood cell subgroups calculated based on the proportions is shown on the left side of the pie chart. (B) in FIG. 11 is a pie chart showing proportions of monocytes and lymphocyte subgroups of the subject before NK cells are administered. (C) in FIG. 11 is a pie chart showing the proportion of only the lymphocyte subgroups of the subject before NK cells are administered.

(D) in FIG. 11 is a pie chart showing proportions of components of the white blood cell subgroups of the subject after NK cells are administered and a calculation example of the number of white blood cell subgroups calculated based on the proportions is shown on the right side of the pie chart. (E) in FIG. 11 is a pie chart showing proportions of monocytes and lymphocyte subgroups of the subject after NK cells are administered. (F) in FIG. 11 is a pie chart showing the proportion of only the lymphocyte subgroups of the subject after NK cells are administered.

As shown in FIG. 11, even after autologous NK cells are injected into health persons 3 times, there is no Th1 immparty amplification phenomenon (CD8↑), and instead, an increasing tendency in the number of immune central lymphocytes CD4 (helper T cells) can be confirmed.

That is, the multi-parameter automatic blood cell counting device 1 of the present embodiment can examine the immune status of healthy persons and immediately confirm compensation of the immune status in a dynamic state, and thus is beneficial for protecting health.

In addition, it is possible to immediately determine the immune status of influenza patients based on the multi-parameter automatic blood cell counting device of the present embodiment.

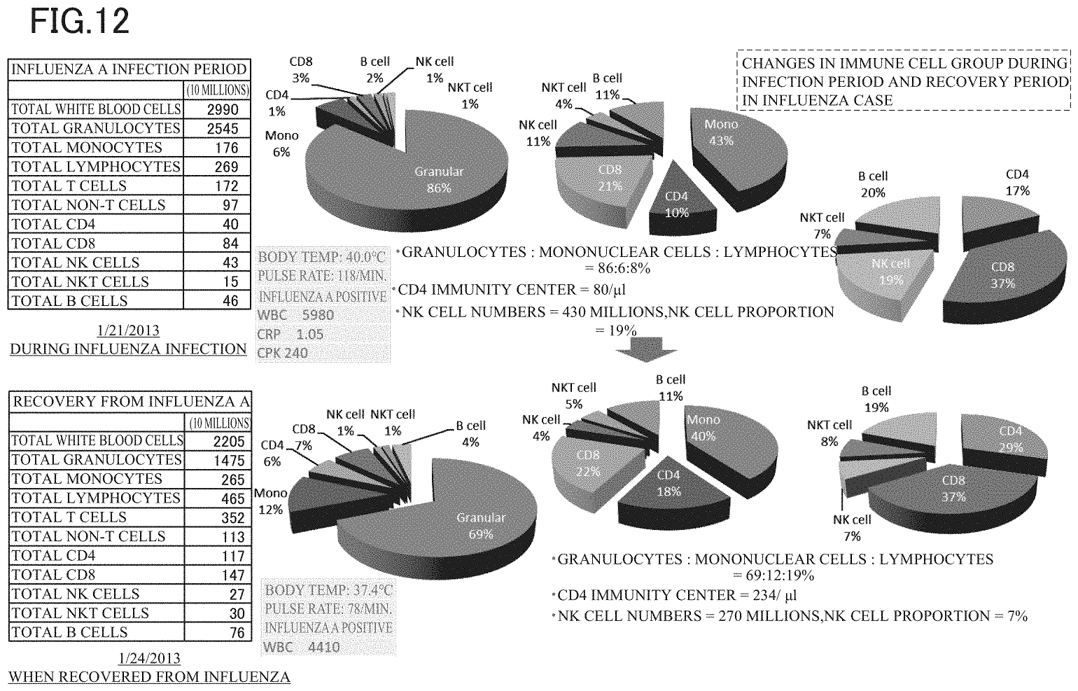

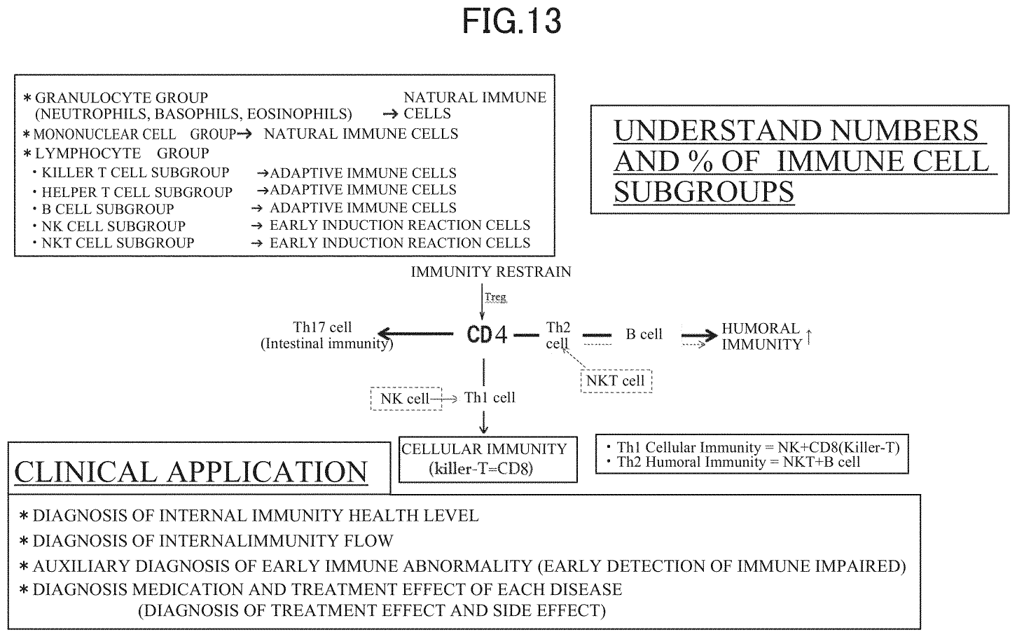

The information displayed by the immunodynamic correlation part 402 is not limited to Output Example 4, but can be used to display an immparty correlation from an influenza pattern 1 to a recovery pattern 1 as shown in FIG. 12. The recovery pattern 1 shows an immune status when the health condition of the influenza patient is recovered by treatment.

As shown in FIG. 12, in acute viral infection such as an influenza infectious disease, at the early stage, a maintenance tendency of NK cells (slow tendency of increase to decrease) and a significant decrease of CD4 cells are observed. On the other hand, during a recovery period, a decrease in the number of NK cells and an increase in the number of CD4 cells are observed. An amplification phenomenon (Booster role) of Th1 cell-mediated immparty due to the increase in the number of NK cells at the early stage of infection suggests an early initiation of innate immparty in which infected cells are removed. An increase in the number of CD4 cells during a recovery period is estimated to be caused by initiation of acquired immparty. Therefore, in the case of an acute viral infectious disease, it is suggested that a rapid action of innate immparty by maintaining the number of NK cells is a key for early recovery from infection and avoiding aggravation.

It is said that in vivo immparty against infection takes about one to two weeks from when natural immparty (innate immparty) is initiated until memory of adaptive immparty (acquired immparty) is completed. During this period, the compensatory change in the immune cell subgroups leads to disease recovery. During acute viral infection such as SARS and MERS, the change in the immune cell subgroups is estimated to have a significant effect on prognosis. That is, a speed at which the immune cell group is compensated is estimated to be a critical factor in life or death. In FIG. 12, at the early stage of infection of an influenza infectious disease which is a clinical acute viral infectious disease, an increasing tendency of the number of NK cells and a significant decrease in the number of CD4 cells are observed, and during a recovery period, a decrease in the number of NK cells and an increase in the number of CD4 cells are observed. An amplification phenomenon (Booster role) of Th1 cell-mediated immparty due to the increase in the number of NK cells at the early stage of infection occurs in early initiation of innate immparty in which infected cells are removed, and during a recovery period, an increase in the number of CD4 Helper-T cells is caused by initiation of acquired immparty. Therefore, in the case of an acute viral infectious disease, an increase in the number of NK cells can be used to estimate the change in immune compensation for early recovery from infection and avoiding aggravation in order to remove infected cells which are virus proliferation factories. That is, it is possible to accurately analyze the immune-compensation state (non-onset) and the immune-decompensation state (onset).

As described above, for a certain number of acute viral infectious disease cases, if dynamic related data of the immune cell subgroups is analyzed using the multi-parameter automatic blood cell counting device of the present embodiment, it is possible to create an appropriate treatment method (create a favorable treatment guidance) resulting in favorable prognosis without physical burden and economic burden on patients. That is, it is possible to immediately determine information about three immunodynamics: (1) immune health level (normal/lowered/improved) and balance evaluation of immune autonomic nerves, (2) determination of the direction of immparty (Th1. Th2. Th17), and (3) determination in the change in immune compensation (immune-compensation state), and it is possible to quickly and appropriately treat the diseases.

While embodiments of the present disclosure have been described above in detail with reference to the drawings, the specific configuration is not limited to the embodiments and output examples, and changes in design without departing from the gist and scope of the present disclosure are included in the present disclosure.

What is claimed is:

1. A multi-parameter automatic blood cell counting device, comprising:
a basic information generating part;
an immune cell subgroup detection part;
an immunodynamic change analyzing part;
a storage part; and
a display part, wherein
the basic information generating part is configured to acquire examination information about a blood count of blood components and a white blood cell image and CD classification analysis information about monoclonal antibodies that bind to lymphocyte surface antigens, and is configured to store the acquired examination information and the CD classification analysis information in the storage part,
the immune cell subgroup detection part is configured to detect information about immune cell subgroups required for analyzing an immune status of a subject based on the examination information and the CD classification analysis information, and
the immunodynamic change analyzing part is configured to identify at least information about the change in immunodynamics of the subject based on the information detected by the immune cell subgroup detection part and the examination information and CD classification analysis information stored in the storage part, and is configured to form an image of the identified information to display the image on the display part.

2. The multi-parameter automatic blood cell counting device according to claim 1, wherein
the immune cell subgroup detection part is configured to calculate the number of B cells, the number of NK cells and the number of NKT cells as the information required for analyzing the immune status of the subject and to detect the number of immune lymphocyte subgroups including at least the number of helper T cells, the number of killer T cells, the number of NK cells, the number of NKT cells and the number of B cells and proportions of the immune lymphocyte subgroups.

3. The multi-parameter automatic blood cell counting device according to claim 1, wherein
the immune cell subgroup detection part is configured to detect a total number of white blood cells as immune cells in a body of the subject and the number of each of the immune cell subgroups based on the examination information and the CD classification analysis information, and
the immunodynamic change analyzing part is configured to generate an immune status pattern or an immunodynamic model based on changes in the proportions and numbers of the immune cell subgroups.

4. The multi-parameter automatic blood cell counting device according to claim 1, wherein
the immunodynamic change analyzing part comprises a pattern forming part configured to form an immune status pattern, an immunodynamic correlation part configured to display information about an immunodynamic correlation of the subject with respect to the immune status pattern, and a self-learning part that has a self-learning function of learning according to the blood count and characterizes a correlation of each blood component of the blood counts.

5. The multi-parameter automatic blood cell counting device according to claim 4, wherein
the self-learning part is configured to determine a pattern range indicating characteristics of an infectious disease based on the numbers of total granulocytes, total lymphoid cells, CD4 antigens, CD8 antigens, NK cells, NKT cells and B cells stored in the storage part and stores the range in the storage part.

6. The multi-parameter automatic blood cell counting device according to claim 5, wherein
the pattern forming part is configured to form an image of the proportions of the number of CD4 antigens, the number of CD8 antigens, the number of NK cells, the number of NKT cells and the number of B cells of an infected person stored in the storage part and to display the image on the display part, and
wherein information for determining whether the subject is infected with influenza using comparison influenza infection pattern standard data stored in the storage part is displayed on the display part.

7. The multi-parameter automatic blood cell counting device according to claim 1, wherein
the immunodynamic change analyzing part is configured to analyze information about degrees of dominance of Th1 cell-mediated immparty and Th2 humoral immparty for acquired immparty of the subject based on the identified information about the change in immunodynamics of the subject.

8. The multi-parameter automatic blood cell counting device according to claim 1, wherein
the immunodynamic change analyzing part is configured to analyze an immune-compensation state and an immune-decompensation state for a clinical status of the subject based on the identified information about the change in immunodynamics of the subject.

9. The multi-parameter automatic blood cell counting device according to claim 1, wherein
the immunodynamic change analyzing part is configured to analyze degrees of dominance of sympathetic and parasympathetic nerves for autonomic nerves of the subject based on the identified information about the change in immunodynamics of the subject.
